(19) United States
(12) Patent Application Publication
(10) Pub. No.: US 2005/0165033 A1
(43) Pub. Date: Jul. 28, 2005

(54) Use of indole-3-acetic acids in the treatment of asthma, copd and other diseases

(76) Inventors: Andrew Baxter, Leicestershire (GB); John Steele, Leicestershire (GB); Simon Teague, Leicestershire (GB)

Correspondence Address: FISH & RICHARDSON P.C., 225 FRANKLIN STREET, BOSTON, MA, 02110, US

(21) Appl. No.: 10/503,708

(22) PCT Filed: Feb. 4, 2003
(86) PCT No.: PCT/SE03/00184

(30) Foreign Application Priority Data
Feb. 5, 2002 (SE) 0200356-4

(51) Int. Cl. A61K 31/517; A61K 31/4709

(52) U.S. Cl. 514/266.2; 514/313

(57) ABSTRACT

The invention relates to 1-(quinazolinfyl)- and 1-(quinolin-4-yl)-indole-3-acetic acid derivatives of the general formula and their use in the treatment of respiratory diseases; such as asthma, rhinitis and chronic obstructive pulmonary disease (COPD); and other diseases mediated by prostaglandin D2 (PGD₂).

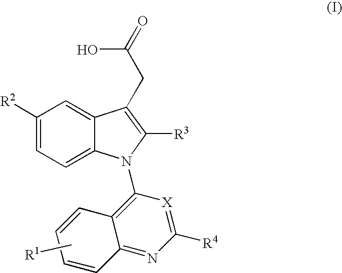

The present invention relates to a new pharmaceutical use for certain indole acetic acids.

EPA 1 170 594 discloses methods for the identification of compounds useful for the treatment of disease states mediated by prostaglandin D2, a ligand for orphan receptor CRTH2. GB 1356834 discloses a series of compounds said to possess anti-inflammatory, analgesic and antipyretic activity. It has now surprisingly been found that certain compounds within the scope of GB 1356834 are active at the CRTH2 receptor, and as a consequence are expected to be potentially useful for the treatment of various respiratory diseases, including asthma and COPD.

In a first aspect the invention therefore provides the use of a compound of formula (I) or a pharmaceutically acceptable salt thereof in the treatment of asthma and COPD: 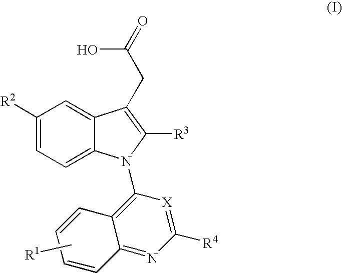
in which
R¹ is hydrogen, halogen, C₁₋₆alkyl, C₁₋₆alkoxy
R² is hydrogen, halogen, C₁₋₆alkyl, C₁₋₆alkoxy
R³ is hydrogen, C₁₋₆alkyl;
R⁴ is hydrogen, C₁₋₆alkyl, C₁₋₆alkoxy, thioC₁₋₆alkyl; and
X is N or CH.

The term alkyl, whether alone or as part of another group, includes straight chain and branched chain alkyl groups.

Preferably R¹ is hydrogen, chloro or methyl.

Preferably R² is methyl, iso-propyl or methoxy.

Preferably R⁴ is hydrogen or methoxy.

Preferably X is CH.

Preferred compounds of the invention include:
1-(7-chloro-4-quinazolinyl)-2-methyl-1H-indole-3-acetic acid
5-methoxy-2-methyl-1-(4-quinazolinyl)-1H-indole-3-acetic acid
2-methyl-1-(2-methyl-4-quinazolinyl)-1H-indole-3-acetic acid
1-(6-chloro-2-quinolinyl)-5-methoxy-2-methyl-1H-indole-3-acetic acid
1-(6,8-dichloro-4-quinazolinyl)-5-methoxy-2-methyl-1H-indole-3-acetic acid
1-(4-chloro-2-quinolinyl)-5-methoxy-2-methyl-1H-indole-3-acetic acid
1-(7-chloro-4-quinazolinyl)-5-methoxy-2-methyl-1H-indole-3-acetic acid
2,5-dimethyl-1-(4-quinazolinyl)-1H-indole-3-acetic acid
1-(7-chloro-4-quinazolinyl)-5-methoxy-2-methyl-1H-indole-3-acetic acid
2,5-dimethyl-1-(4-quinazolinyl)-1H-indole-3-acetic acid
1-(7-chloro-4-quinazolinyl)-5-fluoro-1H-indole-3-acetic acid
5-methoxy-2-methyl-1-[2-(methylthio)-4-quinazolinyl]-1H-indole-3-acetic acid
5-methoxy-1-(6-methoxy-4-quinolinyl)-2-methyl-1H-indole-3-acetic acid
2-methyl-1-[2-(methylthio)-4-quinazolinyl]-1H-indole-3-acetic acid
1-[2-(ethylthio)-4-quinazolinyl]-5-methoxy-2-methyl-1H-indole-3-acetic acid
1-(7-chloro-4-quinazolinyl)-2,5-dimethyl-1H-indole-3-acetic acid
1-(7-chloro-4-quinazolinyl)-2-methyl-5-(1-methylethyl)-1H-indole-3-acetic acid
2,5-dimethyl-1-[2-(methylthio)-4-quinazolinyl]-1H-indole-3-acetic acid
1-(7-chloro-4-quinazolinyl)-2-methyl-5-(2-methylpropoxy)-1H-indole-3-acetic acid
1-(7-chloro-4-quinolinyl)-2-methyl-1H-indole-3-acetic acid
1-(7-chloro-2-methyl-4-quinolinyl)-5-methoxy-2-methyl-1H-indole-3-acetic acid
5-methoxy-2-methyl-1-(7-methyl-4-quinolinyl)-1H-indole-3-acetic acid
1-(7-chloro-4-quinolinyl)-5-methoxy-2-methyl-1H-indole-3-acetic acid
   and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

Certain compounds of formula (I) are believed to be novel and form a further aspect of the invention.

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate orp-toluenesulphonate.

The compounds of formula (I) have activity as pharmaceuticals, in particular as modulators of CRTh2 receptor activity, and may be used in the,treatment (therapeutic or prophylactic) of conditions/diseases in human and non-human animals which are exacerbated or caused by excessive or unregulated production of PGD₂ and its metabolites. Examples of such conditions/diseases include:
(1) (the respiratory tract) obstructive airways diseases including: asthma (such as bronchial, allergic, intrinsic, extrinsic and dust asthma particularly chronic or inveterate asthma (e.g. late asthma and airways hyper-responsiveness)); chronic obstructive pulmonary disease (COPD)(such as irreversible COPD); bronchitis (including eosinophilic bronchitis); acute, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofoulous rhinitis, perennial allergic rhinitis, easonal rhinitis (including rhinitis nervosa (hay fever) and vasomotor rhinitis); nasal polyposis; sarcoidosis; farmer's lung and related diseases; fibroid lung; idiopathic interstitial pneumonia; cystic fibrosis; antitussive activity; treatment of chronic cough associated with inflammation or iatrogenic induced;
(2) (bone and joints) arthrides including rheumatic, infectious, autoimmune, seronegative, spondyloarthropathies (such as ankylosing spondylitis, psoriatic arthritis and Reiter's disease), Behcet's disease, Sjogren's syndrome and systemic sclerosis;
(3) (skin and eyes) psoriasis, atopical dermatitis, contact dermatitis, other eczmatous dermitides, seborrhoetic dernatitis, Lichen planus, Pemphigus, bullous Pemphigus, Epidermolysis bullosa, urticaria, angiodermas, vasculitides, erythemas, cutaneous eosinophilias, chronic skin ulcers, uveitis, Alopecia areatacomeal ulcer and vernal conjunctivitis;
(4) (gastrointestinal tract) Coeliac disease, proctitis, eosinopilic gastro-enteritis, mastocytosis, Crohn's disease, ulcerative colitis, irritable bowel disease; food-related allergies which have effects remote from the gut, (such as migraine, rhinitis and eczema);
(5) (central and peripheral nervous system) Neurodegenerative diseases and dementia disorders (such as Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia), polyneuropathies (such as Guillain-Barré syndrome, chronic inflammatory demyelinating polyradiculoneuropathy, multifocal motor neuropathy), plexopathies, CNS demyelination (such as multiple sclerosis, acute disseminated/haemorrhagic encephalomyelitis, and subacute sclerosing panencephalitis), neuromuscular disorders (such as myasthenia gravis and Lambert-Eaton syndrome), spinal diorders (such as tropical spastic paraparesis, and stiff-man syndrome), paraneoplastic syndromes (such as cerebellar degeneration and encephalomyelitis), CNS trauma, migraine and stroke.
(6) (other tissues and systemic disease) atherosclerosis, Acquired Immunodeficiency Syndrome (AIDS), lupus erythematosus; systemic lupus, erythematosus; Hashimoto's thyroiditis, type I diabetes, nephrotic syndrome, eosinophilia fascitis, hyper IgE syndrome, lepromatous leprosy, idiopathic thrombocytopenia pupura; post-operative adhesions, sepsis and ischemic/reperfusion injury in the heart, brain, peripheral limbs hepatitis (alcoholic, steatohepatitis and chronic viral), glomerulonephritis, renal impairment, chronic renal failure and other organs
(7) (allograft rejection) acute and chronic following, for example, transplantation of kidney, heart, liver, lung, bone marrow, skin and cornea; and chronic graft versus host disease;
(8) Diseases associated with raised levels of PGD₂ or its metabolites.

Thus, the present invention provides a compound of formula (I), or a pharmaceutically-acceptable salt or solvate thereof, as hereinbefore defined for use in therapy.

Preferably the compounds of the invention are used to treat diseases in which the chemokine receptor belongs to the CRTh2 receptor subfamily.

Particular conditions which can be treated with the compounds of the invention are asthma, rhinitis and other diseases in which raised levels of PGD₂ or its metabolites. It is preferred that the compounds of the invention are used to treat asthma.

In a further aspect, the present invention provides the use of a compound of formula (I), or a pharmaceutically acceptable salt or solvate thereof, as hereinbefore defined in the manufacture of a medicament for use in therapy.

In a further aspect, the present invention provides the use of a compound or formula (I), or a pharmaceutically acceptable salt or solvate thereof, as hereinbefore defined in the manufacture of a medicament for use in therapy in combination with drugs used to treat asthma and rhinitis (such as inhaled and oral steroids, inhaled β2-receptor agonists and oral leukotriene receptor antagonists).

In a further aspect, the present invention provides the use of a compound of formula (I), or a pharmaceutically acceptable salt or solvate thereof, as hereinbefore defined in the manufacture of a medicament for use in therapy.

The invention still further provides a method of treating a disease mediated by prostaglandin D2, which comprises administering to a patient a therapeutically effective amount of a compound of formula (I), or a pharmaceutically acceptable salt or solvate thereof, as hereinbefore defined.

The invention also provides a method of treating a respiratory disease, such as athma and rhinitis, especially asthma, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

### EXPERIMENTAL SECTION

Compounds of formula (I) can be prepared according to the procedures outlined in GB 1356834.

Pharmacological Data—Intracellular Calcium Mobilisation

Human Embryonic Kidney Cells co-transfected with both the CRTh2 receptor and GO 16 G-protein (HEK-hrCRTh2-G□16) are routinely cultured as monolayers in Dulbecco's Modified Eagles Medium (DMEM; Sigma) supplemented with 10% (v:v) heat inactivated foetal bovine serum (New Zealand sourced; Hyclone), 1% (v:v) non-essential amino acids (Gibco BRL), 1% (v:v) penicillin/streptomycin (Gibco BRL), 2 mM L-glutamine (Gibco BRL) and grown under 1 mg/ml (v:v) Geneticin (Gibco BRL) antibiotic selection. Approximately 24 hours prior to the assay the cells are plated at a seeding density of 100,000 cells/well in 100 □l growth media into black walled 96 well Poly-D-Lysine coated plates (Becton Dickinson), with clear bottoms to allow cell inspection and fluorescence measurements from the bottom of each well. All cultures are maintained under standard tissue culture conditions (37° C. in a humidified atmosphere of 5% CO₂).

To enable changes in intracellular calcium levels to be measured in HEK-hrCRTh2-Gα16 cells fluo-3AM is utilised as the fluorescent calcium indicator. A dye loading buffer is prepared which consists of a final concentration of 5 □M Fluo-3AM fluorescent cytoplasmic calcium indicator dye (Tef Labs), 2.2 □l/ml Pluronic F127 (Molecular Probes) to promote dye uptake, and 0.5 mM brilliant black (Sigma) to reduce background fluorescence in Balanced Salt Solution (BSS; 125 mM NaCl, 5.4 mM KCl, 16.2 mM NaHCO₃, 0.8 mM MgCl₂, 1 mM CaCl₂, 20 mM HEPES, 1 mM NaH₂PO4, 5.5 mM D-(+)-Glucose, 0.1% BSA and pH 7.4 with NaOH). On the day of the assay, the cells are dye loaded in the dark for 60 min at 37° C. by removing the existing growth media and adding 100 □l of the dye loading buffer to each well.

Test compounds are made up at a stock concentration of 10 mM in DMSO. The compounds to be evaluated are then prepared, by serial dilution in BSS buffer, to the required concentrations for inhibition dose response curves to be constructed. These dilutions are then placed into the 1^{st} addition plate which is pre-warmed to 37° C. prior to assay. A PGD₂ (Cayman Chemical) E/[A] curve is generated for each independent assay by measuring the flux of intracellular calcium in response to increasing agonist concentrations. This allows the potency agonist (p[A]₅₀) value to be determined for the HEK-hrCRTh2-Gα16 cells on any given day. Once the p[A]₅₀ for PGD₂ has been determined a separate assay plate containing 2×p[A]₅₀ of PGD₂ is prepared as the 2^{nd} addition plate (or agonist plate). This PGD₂ plate is also pre-warmed to 37° C. prior to assay. The inhibition curve data obtained is then fitted as described below to estimate an IC₅₀ value (concentration of the test compound which produces 50% inhibition of the response to PGD₂).

Measurements of increases in intracellular Ca²⁺ ([Ca²⁺]ᵢ) are then made using a 96 well FLIPr. Fluorescence changes are measured after the addition of either the test AR-C compound on its own (1^{st} addition plate) or the test compound (1^{st} addition plate) followed by the reference agonist, PGD₂ (2^{nd} addition plate).

Measurements of increases in intracellular Ca²⁺ ([Ca²⁺]ᵢ) are then made with the laser intensity set to a suitable level to obtain basal values of approximately 10,000 fluorescence units. To asses compound activity alone fluorescence readings are measured over 5 minutes (1^{st} plate addition), then agonist is added and the compound activity in competition is assessed for a further 2 minutes. The maximum fluorescent signal generated by PGD₂ is typically greater than 15,000 units and obtained with 15 sec of addition.

Agonist Analysis:

Absolute fluorescence units for PGD₂ control E/[A] curve data are fitted to the following form of the Hill equation using a 4 parameter logistic curve fitting program,

$$E = • + \frac{{( \tilde{•} • ) [ A ]}^{m}}{{[ A ]}^{m} + {[ {A}_{50} ]}^{m}} \qquad Equation ( 1 )$$

in which □ and □ are the upper and lower asymptote respectively, and [A]₅₀ and m are the location and slope parameters respectively. Using the calculated □ value, the absolute fluorescence units were subsequently expressed as a % of this value. For AR-C compounds that displayed agonism, the p[A]₅₀ was estimated as well as the intrinsic acitivity (□ of test agonlist/□ of PGD₂).
Antagonist Analysis:

Antagonist affinity values were estimated using the pIC₅₀ Cheng-Prusoff analysis. To this end a PGD₂ E/[A] curve was constructed (see above) and fitted to equation 1 to estimate the potency ([A]₅₀]) and slope (m) values. The effects of the test compound were then assessed against 2×p[A]₅₀ concentration of the reference agonist, PGD₂. The inhibition curve data obtained was subsequently fitted to equation 1 to estimate an IC₅₀ value (concentration of the test compound which produces 50% inhibition of the response to PGD₂).

Compounds of formula (I) have a IC₅₀ value of less than (<) 10 μM.

Specifically 1-(7-chloro-4-quinazolinyl)-2-methyl-1H-indole-3-acetic acid has a pA₂=5.8, 1-(6,8-dichloro-4-quinazolinyl)-5-methoxy-2-methyl-1H-indole-3-acetic acid has a pA₂=6.0 and 1-(7-chloro-4-quinazolinyl)-2-methyl-5-(1-methylethyl)-1H-indole-3-acetic acid has a pA₂=6.8

1. A method, comprising:
treating asthma or COPD in a patient by administering to the patient a therapeutically effective amount of a compound of formula (I) or a pharmaceutically acceptable salt thereof: 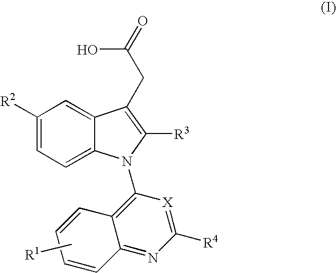
in which
R¹ is hydrogen, halogen, C₁₋₆alkyl, C₁₋₆alkoxy
R² is hydrogen, halogen, C₁₋₆alkyl, C₁₋₆alkoxy
R³ is hydrogen, C₁₋₆alkyl;
R⁴ is hydrogen, C₁₋₆alkyl, C₁₋₆alkoxy, thioC₁₋₆alkyl; and
X is N or CH.

2. The method according to claim 1 in which R¹ is hydrogen, chloro or methyl.

3. The method according to claim 1 in which R² is methyl, iso-propyl or methoxy.

4. The method according to claim 1 in which R⁴ is hydrogen or methoxy.

5. The method according to claim 1 in which X is CH.

6. The method according to claim 1 in which the compound of formula (I) is selected from:
1-(7-chloro-4-quinazolinyl)-2-methyl-1H-indole-3-acetic acid
5-methoxy-2-methyl-1-(4-quinazolinyl)-1H-indole-3-acetic acid
2-methyl-1-(2-methyl-4-quinazolinyl)-1H-indole-3-acetic acid
1-(6-chloro-2-quinolinyl)-5-methoxy-2-methyl-1H-indole-3-acetic acid
1-(6,8-dichloro-4-quinazolinyl)-5-methoxy-2-methyl-1H-indole-3-acetic acid
1-(4-chloro-2-quinolinyl)-5-methoxy-2-methyl-1H-indole-3-acetic acid
1-(7-chloro-4-quinazolinyl)-5-methoxy-2-methyl-1H-indole-3-acetic acid
2,5-dimethyl-1-(4-quinazolinyl)-1H-indole-3-acetic acid
1-(7-chloro-4-quinazolinyl)-5-methoxy-2-methyl-1H-indole-3-acetic acid
2,5-dimethyl-1-(4-quinazolinyl)-1H-indole-3-acetic acid
1-(7-chloro-4-quinazolinyl)-5-fluoro-1H-indole-3-acetic acid
5-methoxy-2-methyl-1-[2-(methylthio)-4-quinazolinyl]-1H-indole-3-acetic acid
5-methoxy-1-(6-methoxy-4-quinolinyl)-2-methyl-1H-indole-3-acetic acid
2-methyl-1-[2-(methylthio)-4-quinazolinyl]-1H-indole-3-acetic acid
1-[2-(ethylthio)-4-quinazolinyl]-5-methoxy-2-methyl-1H-indole-3-acetic acid
1-(7-chloro-4-quinazolinyl)-2,5-dimethyl-1H-indole-3-acetic acid
1-(7-chloro-4-quinazolinyl)-2-methyl-5-(1-methylethyl)-1H-indole-3-acetic acid
2,5-dimethyl-1-[2-(methylthio)-4-quinazolinyl]-1H-indole-3-acetic acid
1-(7-chloro-4-quinazolinyl)-2-methyl-5-(2-methylpropoxy)-1H-indole-3-acetic acid
1-(7-chloro-4-quinolinyl)-2-methyl-1H-indole-3-acetic acid
1-(7-chloro-2-methyl-4-quinolinyl)-5-methoxy-2-methyl-1H-indole-3-acetic acid
5-methoxy-2-methyl-1-(7-methyl-4-quinolinyl)-1H-indole-3-acetic acid
1-(7-chloro-4-quinolinyl)-5-methoxy-2-methyl-1H-indole-3-acetic acid
and pharmaceutically acceptable salts thereof.

7. (canceled)

8. A method, comprising:
treating a disease mediated by prostaglandin D2, by administering to a patient a therapeutically effective amount of a compound of formula (I), 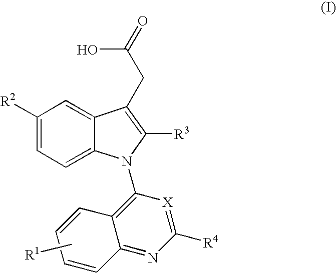
in which
R¹ is hydrogen, halogen, C₁₋₆alkyl, C₁₋₆alkoxy
R² is hydrogen, halogen C₁₋₆alkyl, C₁₋₆alkoxy
R³ is hydrogen, C₁₋₆alkyl;
R⁴ is hydrogen, C₁₋₆alkyl, C₁₋₆alkoxy, thioC₁₋₆alkyl, and
X is N or CH,
or a pharmaceutically acceptable salt thereof.

9. A method, comprising:
treating a respiratory disease, such as asthma and rhiritis, in a patient suffering from, or at risk of, said disease, which rises by administering to the patient a therapeutically effective amount of a compound of formula (I), 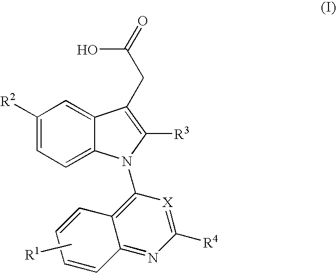
in which
R¹ is hydrogen, halogen, C₁₋₆alkyl, C₁₋₆alkoxy
R² is hydrogen, halogen, C₁₋₆alkyl, C₁₋₆alkoxy
R³ is hydrogen, C₁₋₆alkyl;
R⁴is hydrogen, C₁₋₆alkyl, C₁₋₆alkoxy, thioC₁₋₆alkyl; and
X is N or CH;
or a pharmaceutically acceptable salt or solvate thereof.

10. The method of claim 9 wherein the respiratory disease is asthma.

11. The method of claim 9 wherein the respiratory disease is rhinitis.
